(19) United States
(12) Patent Application Publication
(10) Pub. No.: US 2025/0157589 A1
(43) Pub. Date: May 15, 2025

(54) COMPOUND IDENTIFICATION USING A MASS SPECTRUM

(71) Applicant: DH TECHNOLOGIES DEVELOPMENT PTE. LTD., Singapore (SG)

(72) Inventors: David Michael Cox, Concord (CA); Gordana Ivosev, Concord (CA)

(21) Appl. No.: 18/835,672

(22) PCT Filed: Feb. 2, 2023
(86) PCT No.: PCT/IB2023/050933

§ 371 Date: Aug. 2, 2024

Related U.S. Application Data
(60) Provisional application No. 63/267,558, filed on Feb. 4, 2022.

(51) Int. Cl. G16C 20/20; H01J 49/00

(52) U.S. Cl. CPC G16C 20/20; H01J 49/0036

(57) ABSTRACT

A measured mass spectrum and intensity data provided as a function of m/z and at least one additional dimension are received. Peaks of the measured spectrum are compared to peaks of each of a plurality of library mass spectra. A set of library mass spectra is identified using a fit score. For each spectrum of the set, a group of related peaks of the measured spectrum calculated using a deconvolution algorithm is recalculated. The recalculation lowers a threshold for selection in the group if a matching peak of the library spectrum contributed to the fit score. A group of related peaks of the measured spectrum is produced for each library spectrum. For each spectrum of the set, peaks of the group are compared to peaks of the library spectrum and a purity score is calculated. At least one library spectrum of the set with the highest purity score is identified.

### CROSS REFERENCE TO RELATED APPLICATIONS

This application claims the benefit of the PCT application having International Application No. PCT/IB2023/050933, filed Feb. 2, 2023, and titled “COMPOUND IDENTIFICATION USING A MASS SPECTRUM,” which in turn claims priority to U.S. Provisional Patent Application Ser. No. 63/267,558, filed on Feb. 4, 2022, the contents of which are incorporated by reference herein in their entirety.

### BACKGROUND

The teachings herein relate to identifying a library mass spectrum of a known compound that matches a measured mass spectrum. More particularly the teachings herein relate to systems and methods for identifying a subset of spectra of a library of mass spectra that match a measured mass spectrum using a fit score, recalculating a group of correlated peaks found from a deconvolution algorithm for each spectrum of the set that varies the confidence threshold for each ion based on the fit score, comparing the recalculated correlated peaks of the measured spectrum found for each library spectrum of with the peaks of the library spectrum using a purity score, and identifying the library spectrum with the best purity score as matching the measured spectrum.

The systems and methods herein can be performed in conjunction with a processor, controller, or computer system, such as the computer system of FIG. 1.

### One-Way Compound Identification Workflow

A one-way mass spectrometry compound identification workflow typically involves comparing a measured product ion spectrum to a library of product ion mass spectra of known compounds or to computer-generated product ion mass spectra generated from a database of known compounds. A known compound whose library mass spectrum or computer-generated mass spectrum matches the measured spectrum the best is then selected as the identified compound.

A one-way compound identification workflow works well for data from an information-dependent acquisition (IDA) or a data-dependent acquisition (DDA) mass spectrometry method. However, this type of compound identification presents several challenges and disadvantages for data from a data-independent acquisition (DIA) mass spectrometry method, often leading to false-positive or false-negative results. More about IDA, DDA, and DIA is provided below.

One challenge presented by DIA methods is that the precursor ion (Q1) mass filter is not as specific as the precursor ion mass filter in an IDA method. This reduced specificity results in measured spectra that are a mixture of product ions from multiple different precursor ions. As a result, product ion spectra from DIA methods require that a deconvolution algorithm be performed before a library or database search is done. In addition, no deconvolution algorithm provides the correct solution 100% of the time.

A first disadvantage presented by DIA methods is that they are typically performed along one or more dimensions in addition to intensity and mass to charge ratio (m/z). These one or more dimensions include, but are not limited to, retention time, mobility separation, and scanning precursor ion m/z. As a result, in DIA deconvolution, intensity is not correlated with deconvolution accuracy.

In other mass spectrometry methods, deconvoluted product ion data typically includes a probability assigned to each m/z peak and correlated to a deconvolution result uncertainty. Instead, in DIA deconvolution, product ions in a resulting product ion spectrum are selected based on some binary criteria (belongs yes or no). Also, all product ions that are included are treated by the compound identification tool with equal relevance (sometimes more intense ones are given some advantage, but this is not correlated with deconvolution accuracy).

Another disadvantage presented by DIA methods is that it is difficult to confidently identify a compound from a product ion spectrum using library searching. In library searching, a fit score is a measure of how well a library spectrum matches the measured spectrum. The fit score is not impacted by additional peaks in the measure spectrum that do not match a peak in the library spectrum. The range of the fit score is 0 to 1.

A fit score, for example, is high (closer to 1) if the peaks in the library spectrum are found in the measured spectrum. Conversely, the fit score is low (closer to 0) if the peaks in the library spectrum are missing from the unknown spectrum. However, if the unknown spectrum has additional peaks (from interfering compounds in the same isolation window), these extra fragment peaks have no impact on the fit score. A drawback then of using the fit score on its own is that it can lead to a high false-positive rate of identification. Essentially, a false-positive is a high score for incorrect identification.

Another type of score used in library searching to identify a compound from a product ion spectrum is a purity score. A purity score is a measure of how well the measured spectrum and the library spectrum match each other. All peaks from both spectra are used. The purity score also ranges from 0 to 1.

A purity score is high (closer to 1), for example, if the peaks in the library spectrum are found in the measured, and there are few or no additional peaks from interfering compounds. If the measured spectrum has additional peaks (from interfering compounds in the same isolation window), the purity score is low (closer to 0). A drawback of using the purity score on its own is that it can miss identification of compounds when there are some interference peaks. A raw DIA product ion spectrum may contain fragments from several compounds due to the large isolation window used in DIA. This leads to poor purity scores and incorrect identifications.

Also, in some cases, DIA data is background-subtracted. With background-subtracted DIA spectra, closely eluting isomers (e.g., morphine and hydromorphone) have similar fragments and the background subtraction erroneously eliminates the signal from these fragments. This leads to poor fit and purity scores.

As described above, to handle mass spectra that include product ions from multiple different precursor ions, DIA methods typically require the use of a deconvolution algorithm. An exemplary unsupervised clustering algorithm, deconvolution algorithm, linear mapping algorithm, or nonlinear mapping algorithm is principal component analysis with variable grouping (PCVG). More about PCVG is provided below.

PCVG is, for example, used to process DIA spectra. PCVG groups together product ions that have a similar extracted ion chromatogram (XIC) profile. PCVG helps remove product ions from interfering compounds and leads to more accurate purity scores. However, ultimately PCVG calculates a confidence value (based on correlation in principal component (PC) space) that a product ion belongs in the same group.

Often a confidence threshold is applied to obtain a spectrum from PCVG analyzed DIA spectra. This threshold is typically the same for all data. The PCVG algorithm maps groups between or within a group variance on the same scale. A group variance is, for example, an average difference between product ions of different components versus an average difference among product ions of the same component. The selected threshold value corresponds to the optimal group variance ratio regardless of data complexity and noise level. The confidence threshold is selected to be sensitive enough to detect small differences in precursor liquid chromatography (LC) profiles (minimize false-negatives) but not to split product ions due to LC differences caused by noise (minimize false-positives).

The use of the same confidence threshold for PCVG works well on product ions that are unique for the precursors within an isolation m/z filter window and an LC-peak region. However, shared product ion LC profiles are a linear combination of the corresponding precursor profiles and, therefore, may not pass the confidence threshold. Thus, shared product ions between two compounds or very closely eluting compounds can lead to incorrect spectra and poor fit and purity scores.

The confidence threshold is just one of the PCVG parameters that can be used. It is just a parameter that is applied at the last stage of clustering. Various embodiments described herein are applicable to any parameter. For example, in particular for PCVG, the number of PCs is determined through a variance explained threshold. Changing this parameter results in a more stringent fragment LC profile (or what is that “other than mz dimension”) similarity requirement for group members, while having less PCs (different variance threshold) results in grouping that tolerates some non-ideal similarities.

Another problem leading to poor fit and purity scores could be noise. The PCVG parameters and the confidence threshold are generally optimized to work with noisy data, but are still designed to be sensitive enough to detect highly similar yet different LC peak profiles. It can happen that ions from same precursor, due to noise, have small LC peak differences that would be identified by PCVG as similar but different groups of peaks, and would be split in two close groups.

PCVG has a limitation compared to some other deconvolution techniques (like NonNegative decomposition methods which can separate shared fragments and assign a fraction of observed intensity to two or more groups). However, those other deconvolution techniques methods have a problem with noise and can also “oversplit” the observed intensity into too many groups and also assign an incorrect group. In most general terms, any deconvolution algorithm has sensitivity/specificity trade off that can result in incomplete or interfered resulting deconvoluted spectrum.

As mentioned above, the key is to identify compounds in two different ways. Any separation method can be used.

In some cases, precursor ion analysis (MS analysis) is also used. MS analysis is used to reduce the number of possible library spectra to be considered, which reduces time and possible false positives.

In general, additional systems and methods are needed to optimize a parameter of an unsupervised clustering algorithm, deconvolution algorithm, linear mapping algorithm, or nonlinear mapping algorithm to improve compound identification in mass spectrometry methods like DIA.

Again, note that PCVG is just one example of an unsupervised clustering algorithm. Also, note that the confidence threshold is just one example of a parameter optimization challenge or limitation associated with an unsupervised clustering algorithm, deconvolution algorithm, linear mapping algorithm, or nonlinear mapping algorithm.

### LC-MS and LC-MS/MS Background

Mass spectrometry (MS) is an analytical technique for the detection and quantitation of chemical compounds based on the analysis of mass-to-charge ratios (m/z) of ions formed from those compounds. The combination of mass spectrometry (MS) and liquid chromatography (LC) is an important analytical tool for the identification and quantitation of compounds within a mixture. Generally, in liquid chromatography, a fluid sample under analysis is passed through a column filled with a chemically-treated solid adsorbent material (typically in the form of small solid particles, e.g., silica). Due to slightly different interactions of components of the mixture with the solid adsorbent material (typically referred to as the stationary phase), the different components can have different transit (elution) times through the packed column, resulting in separation of the various components.

Note that the terms “mass” and “m/z” are used interchangeably herein. One of ordinary skill in the art understands that a mass can be found from an m/z by multiplying the m/z by the charge. Similarly, the m/z can be found from a mass by dividing the mass by the charge.

In LC-MS, the effluent exiting the LC column can be continuously subjected to MS analysis. The data from this analysis can be processed to generate an extracted ion chromatogram (XIC), which can depict detected ion intensity (a measure of the number of detected ions of one or more particular analytes) as a function of retention time.

In MS analysis, an MS or precursor ion scan is performed at each interval of the separation for a mass range that includes the precursor ion. An MS scan includes the selection of a precursor ion or precursor ion range and mass analysis of the precursor ion or precursor ion range.

In some cases, the LC effluent can be subjected to tandem mass spectrometry (or mass spectrometry/mass spectrometry MS/MS) for the identification of product ions corresponding to the peaks in the XIC. For example, the precursor ions can be selected based on their mass/charge ratio to be subjected to subsequent stages of mass analysis. For example, the selected precursor ions can be fragmented (e.g., via collision-induced dissociation), and the fragmented ions (product ions) can be analyzed via a subsequent stage of mass spectrometry.

### Tandem Mass Spectrometry or MS/MS Background

Tandem mass spectrometry or MS/MS involves ionization of one or more compounds of interest from a sample, selection of one or more precursor ions of the one or more compounds, fragmentation of the one or more precursor ions into product ions, and mass analysis of the product ions.

Tandem mass spectrometry can provide both qualitative and quantitative information. The product ion spectrum can be used to identify a molecule of interest. The intensity of one or more product ions can be used to quantitate the amount of the compound present in a sample.

A large number of different types of experimental methods or workflows can be performed using a tandem mass spectrometer. These workflows can include, but are not limited to, targeted acquisition, information dependent acquisition (IDA) or data dependent acquisition (DDA), and data independent acquisition (DIA).

In a targeted acquisition method, one or more transitions of a precursor ion to a product ion are predefined for a compound of interest. As a sample is being introduced into the tandem mass spectrometer, the one or more transitions are interrogated during each time period or cycle of a plurality of time periods or cycles. In other words, the mass spectrometer selects and fragments the precursor ion of each transition and performs a targeted mass analysis for the product ion of the transition. As a result, a chromatogram (the variation of the intensity with retention time) is produced for each transition. Targeted acquisition methods include, but are not limited to, multiple reaction monitoring (MRM) and selected reaction monitoring (SRM).

MRM experiments are typically performed using “low resolution” instruments that include, but are not limited to, triple quadrupole (QqQ) or quadrupole linear ion trap (QqLIT) devices. With the advent of “high resolution” instruments, there was a desire to collect MS and MS/MS using workflows that are similar to QqQ/QqLIT systems. High-resolution instruments include, but are not limited to, quadrupole time-of-flight (QqTOF) or orbitrap devices. These high-resolution instruments also provide new functionality.

MRM on QqQ/QqLIT systems is the standard mass spectrometric technique of choice for targeted quantification in all application areas, due to its ability to provide the highest specificity and sensitivity for the detection of specific components in complex mixtures. However, the speed and sensitivity of today's accurate mass systems have enabled a new quantification strategy with similar performance characteristics. In this strategy (termed MRM high resolution (MRM-HR) or parallel reaction monitoring (PRM)), looped MS/MS spectra are collected at high-resolution with short accumulation times, and then fragment ions (product ions) are extracted post-acquisition to generate MRM-like peaks for integration and quantification. With instrumentation like the TRIPLETOF® Systems of AB SCIEX™, this targeted technique is sensitive and fast enough to enable quantitative performance similar to higher-end triple quadrupole instruments, with full fragmentation data measured at high resolution and high mass accuracy.

In other words, in methods such as MRM-HR, a high-resolution precursor ion mass spectrum is obtained, one or more precursor ions are selected and fragmented, and a high-resolution full product ion spectrum is obtained for each selected precursor ion. A full product ion spectrum is collected for each selected precursor ion but a product ion mass of interest can be specified and everything other than the mass window of the product ion mass of interest can be discarded.

In an IDA (or DDA) method, a user can specify criteria for collecting mass spectra of product ions while a sample is being introduced into the tandem mass spectrometer. For example, in an IDA method a precursor ion or mass spectrometry (MS) survey scan is performed to generate a precursor ion peak list. The user can select criteria to filter the peak list for a subset of the precursor ions on the peak list. The survey scan and peak list are periodically refreshed or updated, and MS/MS is then performed on each precursor ion of the subset of precursor ions. A product ion spectrum is produced for each precursor ion. MS/MS is repeatedly performed on the precursor ions of the subset of precursor ions as the sample is being introduced into the tandem mass spectrometer.

In proteomics and many other applications, however, the complexity and dynamic range of compounds is very large. This poses challenges for traditional targeted and IDA methods, requiring very high-speed MS/MS acquisition to deeply interrogate the sample in order to both identify and quantify a broad range of analytes.

As a result, DIA methods, the third broad category of tandem mass spectrometry, were developed. These DIA methods have been used to increase the reproducibility and comprehensiveness of data collection from complex samples. DIA methods can also be called non-specific fragmentation methods. In a DIA method the actions of the tandem mass spectrometer are not varied among MS/MS scans based on data acquired in a previous precursor or survey scan. Instead, a precursor ion mass range is selected. A precursor ion mass selection window is then stepped across the precursor ion mass range. All precursor ions in the precursor ion mass selection window are fragmented and all of the product ions of all of the precursor ions in the precursor ion mass selection window are mass analyzed.

The precursor ion mass selection window used to scan the mass range can be narrow so that the likelihood of multiple precursors within the window is small. This type of DIA method is called, for example, MS/MS^{ALL}. In an MS/MS^{ALL} method, a precursor ion mass selection window of about 1 Da is scanned or stepped across an entire mass range. A product ion spectrum is produced for each 1 Da precursor mass window. The time it takes to analyze or scan the entire mass range once is referred to as one scan cycle. Scanning a narrow precursor ion mass selection window across a wide precursor ion mass range during each cycle, however, can take a long time and is not practical for some instruments and experiments.

As a result, a larger precursor ion mass selection window, or selection window with a greater width, is stepped across the entire precursor mass range. This type of DIA method is called, for example, SWATH acquisition. In a SWATH acquisition, the precursor ion mass selection window stepped across the precursor mass range in each cycle may have a width of 5-25 Da, or even larger. Like the MS/MS^{ALL} method, all of the precursor ions in each precursor ion mass selection window are fragmented, and all of the product ions of all of the precursor ions in each mass selection window are mass analyzed. However, because a wider precursor ion mass selection window is used, the cycle time can be significantly reduced in comparison to the cycle time of the MS/MS^{ALL} method.

U.S. Pat. No. 8,809,770 describes how SWATH acquisition can be used to provide quantitative and qualitative information about the precursor ions of compounds of interest. In particular, the product ions found from fragmenting a precursor ion mass selection window are compared to a database of known product ions of compounds of interest. In addition, ion traces or extracted ion chromatograms (XICs) of the product ions found from fragmenting a precursor ion mass selection window are analyzed to provide quantitative and qualitative information.

However, identifying compounds of interest in a sample analyzed using SWATH acquisition, for example, can be difficult. It can be difficult because either there is no precursor ion information provided with a precursor ion mass selection window to help determine the precursor ion that produces each product ion, or the precursor ion information provided is from a mass spectrometry (MS) observation that has a low sensitivity. In addition, because there is little or no specific precursor ion information provided with a precursor ion mass selection window, it is also difficult to determine if a product ion is convolved with or includes contributions from multiple precursor ions within the precursor ion mass selection window.

As a result, a method of scanning the precursor ion mass selection windows in SWATH acquisition, called scanning SWATH, was developed. Essentially, in scanning SWATH, a precursor ion mass selection window is scanned across a mass range so that successive windows have large areas of overlap and small areas of non-overlap. This scanning makes the resulting product ions a function of the scanned precursor ion mass selection windows. This additional information, in turn, can be used to identify the one or more precursor ions responsible for each product ion.

Scanning SWATH has been described in International Publication No. WO 2013/171459 A2 (hereinafter “the '459 Application”). In the '459 Application, a precursor ion mass selection window or precursor ion mass selection window of 25 Da is scanned with time such that the range of the precursor ion mass selection window changes with time. The timing at which product ions are detected is then correlated to the timing of the precursor ion mass selection window in which their precursor ions were transmitted.

The correlation is done by first plotting the mass-to-charge ratio (m/z) of each product ion detected as a function of the precursor ion m/z values transmitted by the quadrupole mass filter. Since the precursor ion mass selection window is scanned over time, the precursor ion m/z values transmitted by the quadrupole mass filter can also be thought of as times. The start and end times at which a particular product ion is detected are correlated to the start and end times at which its precursor is transmitted from the quadrupole. As a result, the start and end times of the product ion signals are used to determine the start and end times of their corresponding precursor ions.

### PCVG Background

PCVG is described in U.S. Pat. No. 7,587,285, which is incorporated herein in its entirety. In PCVG, groups of correlated variables are identified using principal component analysis (PCA).

FIG. 2 is an exemplary flowchart showing a method 200 for identifying a group of correlated variables after PCA of a plurality of variables from a plurality of samples using PCVG that is consistent with the present teachings.

In step 210 of method 200, a number of PCs produced by the PCA is selected. The number of PCs selected is, for example, less than the total number of PCs produced by the PCA. In various embodiments, the number of PCs selected is the smallest number that represents a specified percentage of the total variance.

In step 220, a subset PC space having the number of PCs selected is created.

In step 230, a variable is selected in the subset PC space. The variable selected is, for example, the variable that is furthest from the origin.

In step 240, a spatial angle is defined around a vector extending from the origin of the subset PC space to the selected variable.

In step 250, a set of one or more variables in the subset PC space is selected within the spatial angle of the vector. In various embodiments, if one or more variables within the set have a significance value less than a threshold value, then the one or more variables are not selected for the first set. The significance value is a minimum distance parameter, for example. The minimum distance parameter is a minimum distance from the origin, for example.

In step 260, the set is assigned to a group, if the set includes a minimum number of variables. The group identifies correlated variables, for example. The minimum number of variables is the number of correlated variables a group is expected to include, for example. The minimum number of variables can be, for example, one or a number greater than one.

FIG. 3 is an exemplary illustration 300 that shows how a set of one or more variables 340 can be found within a spatial angle 350 of a selected variable 360, in accordance with the present teachings. The three-dimensional PC space shown in FIG. 3 includes PCs PC1 310, PC2 320, and PC3 330. Variable 360 is selected in this three-dimensional PC space. Spatial angle 350 is defined around a vector extending from the origin to selected variable 360. One or more variables found within spatial angle 350 are selected as the set of one or more variables 340. Note that only three dimensions are shown in FIG. 3. In various embodiments, PC space can include more or less than three dimensions. Three dimensions are shown for illustrative purposes.

FIG. 4 is an exemplary schematic diagram showing a computing system 400 for grouping variables after PCA of a plurality of variables from a plurality of samples produced by a measurement technique that is consistent with the present teachings. Computing system 400 includes grouping module 410. Grouping module 410 selects the number of PCs produced by the PCA, creates a subset PC space having the number of PCs, selects a variable, defines a spatial angle around a vector extending from an origin to the variable, selects a set of one or more variables within the spatial angle of the vector, and assigns the set to a group, if the set includes a minimum number of variables.

In various embodiments of computing system 400, the plurality of variables can be generated using a measurement technique that generates more than one variable per constituent of a sample. The plurality of variables is generated using a measurement device. A measurement device can be, but is not limited to, a spectrometer or a mass spectrometer. Measurement techniques can include, but are not limited to, nuclear magnetic resonance, infra-red spectrometry, near infra-red spectrometry, ultra-violet spectrometry, Raman spectrometry, or mass spectrometry. In various embodiments the plurality of variables can be generated using a measurement technique that generates more than one variable per constituent of a sample combined with a separation technique. Separation techniques can include, but are not limited to, liquid chromatography, gas chromatography, or capillary electrophoresis.

In various embodiments, grouping module 410 can also select a second variable in the PC space, select a second set of one or more variables within the spatial angle of a second vector extending from the origin to the second variable, and assign the second set to a second group of variables, if the second set comprises the minimum number of variables.

Another PCVG method consistent with the present teachings is outlined below:
1. Perform PCA on all variables using Pareto scaling. Note that Pareto scaling is just one scaling method. PCVG can use any type of scaling method.
2. Determine the number of PCs (m) to be used. Using all n of the PCs extracted will exactly reproduce the original data. However, many of these PCs represent noise fluctuations in the data and can be ignored with no loss of information. Selecting m PCs effectively smoothes the data. Each variable is represented by a vector in this m-dimensional space.
3. Determine the target vector (t) that corresponds to the variable furthest from the origin. For this to be effective autoscaling is not used. Autoscaling is undesirable because it weights all variables, including small noise peaks, equally.
4. Define a spatial angle (a) around this vector and find other data points (vectors) that are within that angle, optionally ignoring low intensity variables. If a second vector is x, then the angle (θ) between x and the target vector can be found from:

$$x \cdot t = \vert x \vert \vert t \vert cos ( θ )$$

5. Calculate the mean of all selected vectors and repeat step 3 using the new mean vector and assign all selected variables to a group. “Re-centering” in this way fine tunes the orientation of the spatial angle and can be effective if the most intense variable is atypical in some way. For example, the profile may be distorted if the peak is saturated in the most concentrated samples. Since Pareto scaling has been used, calculating the mean vector also causes the lower intensity ions to have less effect on the result.
6. Repeat the process from step 3 ignoring previously grouped variables until there are no remaining variables with sufficient intensity.

FIG. 5 is an exemplary flowchart showing a computer-implemented method 500 that can be used for processing data in n-dimensional space and that is consistent with the present teachings.

In step 510 of method 500, PCA is performed on all variables and the specified subset of PCs is used.

In step 520, variables with low significance are removed. Filtering out variables that have low significance with respect to the selected scaling and PCA significance measure is optional. The same effect can be achieved by adding a step after grouping the variables and by using a different significance criterion. Another significance criterion that can be used is optical contrast, for example. Optical contrast is, for example, only “optical” if the data is presented as an image, but can be applied on a one-dimensional signal as well.

In step 530, a vector of an unassigned variable furthest from the origin is found.

In step 540, all vectors within a spatial angle of the vector are found.

In step 550, a mean of vectors within a spatial angle of the vector is found.

In step 560, all unassigned variables within the spatial angle of the mean are found and assigned to a group. Variables assigned to the group are then removed from processing.

In step 570, if any variables are left for processing, method 500 returns to step 530. If no variables are left for processing, method 500 ends.

The result of this processing is a number of groups of correlated variables that can be interpreted further, or group representations that can be used as input to subsequent analysis techniques. For visualization purposes, it is useful to identify grouped variables in a loadings plot by assigning a symbol to the group. Interpretation can be aided by generating intensity or profile plots for all members of a group. Iterative PCVG is described in U.S. Pat. No. 8,180,581, which is incorporated herein in its entirety.

### SUMMARY

A system, method, and computer program product are disclosed for identifying a library mass spectrum of a known compound that matches a measured mass spectrum. A measured mass spectrum and additional intensity data for ions of the measured mass spectrum as a function of m/z and at least one additional dimension are received from a mass spectrometry method.

Peaks of the measured mass spectrum are compared to peaks of each of a plurality of library mass spectra of different known compounds. A first score is calculated for how well peaks of each library mass spectrum match peaks of the measured mass spectrum. A set of library mass spectra is identified where each mass spectrum of the set has a score above a first score threshold. A group of related peaks of the measured mass spectrum is calculated using an unsupervised clustering algorithm, deconvolution algorithm, linear mapping algorithm, or nonlinear mapping algorithm.

For each library mass spectrum of the set, the group of related peaks of the measured mass spectrum is recalculated. The recalculation of the group uses the additional intensity data and lowers a threshold for selection in the group for a peak of the measured mass spectrum if a matching peak of the library mass spectrum contributed to the first score of the library mass spectrum above a contribution threshold amount.

A corresponding group of related peaks of the measured mass spectrum is produced for each library mass spectrum of the set.

In various embodiments, the similarity threshold is iteratively lowered, which changes the score (both, purity and fit) until either of the scores starts to decline below a threshold. This is done for each library spectrum and results in highest score for each of library spectrum.

For each library mass spectrum of the set, peaks of the corresponding group of the library mass spectrum are compared to peaks of the library mass spectrum and a second score is calculated for how well all peaks of the corresponding group of the library mass spectrum match all peaks of the library mass spectrum. At least one library mass spectrum of the set with the highest second score is identified.

These and other features of the applicant's teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is an exemplary flowchart showing a method for identifying a group of correlated variables after PCA of a plurality of variables from a plurality of samples using PCVG that is consistent with the present teachings.

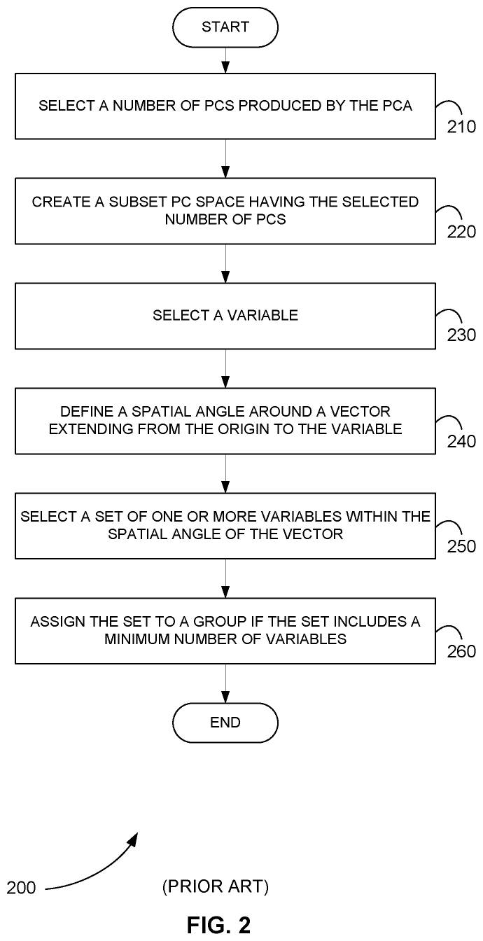

FIG. 3 is an exemplary illustration that shows how a set of one or more variables can be found within a spatial angle of a selected variable, in accordance with the present teachings.

FIG. 4 is an exemplary schematic diagram showing a computing system for grouping variables after PCA of a plurality of variables from a plurality of samples produced by a measurement technique that is consistent with the present teachings.

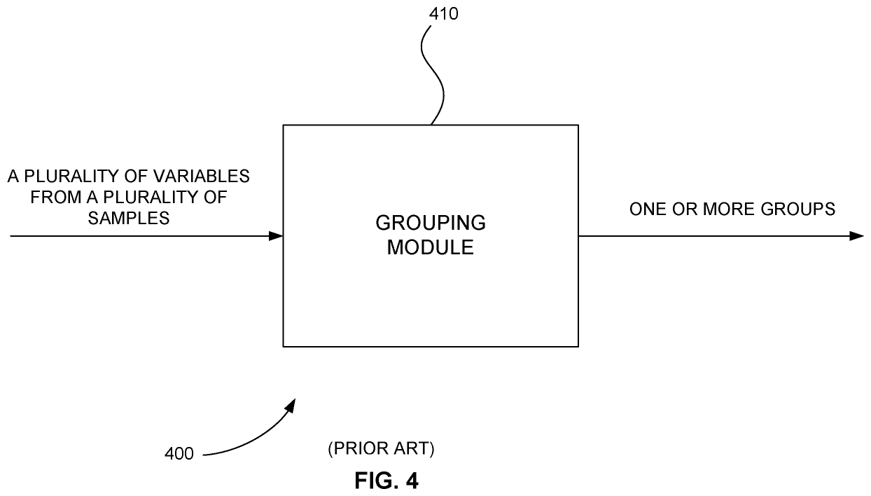

FIG. 5 is an exemplary flowchart showing a computer-implemented method that can be used for processing data in n-dimensional space and that is consistent with the present teachings.

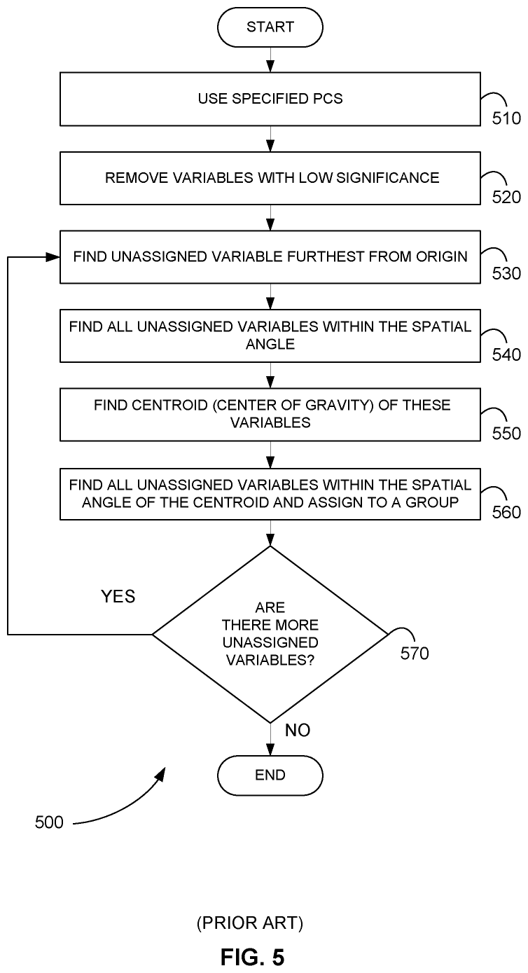

FIG. 6 is an exemplary simplified product ion mass spectrum that is measured during a DIA method, in accordance with various embodiments.

FIG. 7 is an exemplary plot of the XICs of product ions corresponding to the seven peaks shown in FIG. 6, in accordance with various embodiments.

FIG. 8 is an exemplary plot of the product ions of the XICs of FIG. 7 plotted in three-dimensional principal component space after PCVG is performed, in accordance with various embodiments.

FIG. 9 is an exemplary alternative representation of the simplified product ion mass spectrum of FIG. 6 showing that, after PCVG is performed with the same confidence threshold or spatial angle for all data, two peaks of the mass spectrum are identified originating from different compounds, in accordance with various embodiments.

FIG. 10 is a diagram showing a traditional comparison of the peaks of FIG. 9 after PCVG to two library spectra and showing the fit and purity scores of the comparison, in accordance with various embodiments.

FIG. 11 is a diagram showing a comparison of the raw peaks of the measured spectrum of FIG. 6 before PCVG to the peaks of the spectra of a library using only the fit score, in accordance with various embodiments.

FIG. 12 is an exemplary plot of the product ions of the XICs of FIG. 7 plotted in three-dimensional principal component space showing how the confidence threshold is lowered during PCVG grouping to include a highly contributing product ion to the fit score of a particular library spectrum, in accordance with various embodiments.

FIG. 13 is a diagram showing how correlated peaks found through PCVG for a particular library spectrum are then compared to that library spectrum using only a purity score, in accordance with various embodiments.

FIG. 14 is a schematic diagram of a system for identifying a library mass spectrum of a known compound that matches a measured mass spectrum, in accordance with various embodiments.

FIG. 15 is an exemplary flowchart showing a method for identifying a library mass spectrum of a known compound that matches a measured mass spectrum, in accordance with various embodiments.

FIG. 16 is an exemplary flowchart showing a detailed method for identifying a library mass spectrum of a known compound that matches a measured mass spectrum, in accordance with various embodiments.

FIG. 17 is a schematic diagram of a system that includes one or more distinct software modules and that performs a method for identifying a library mass spectrum of a known compound that matches a measured mass spectrum, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DETAILED DESCRIPTION

### Computer-Implemented System

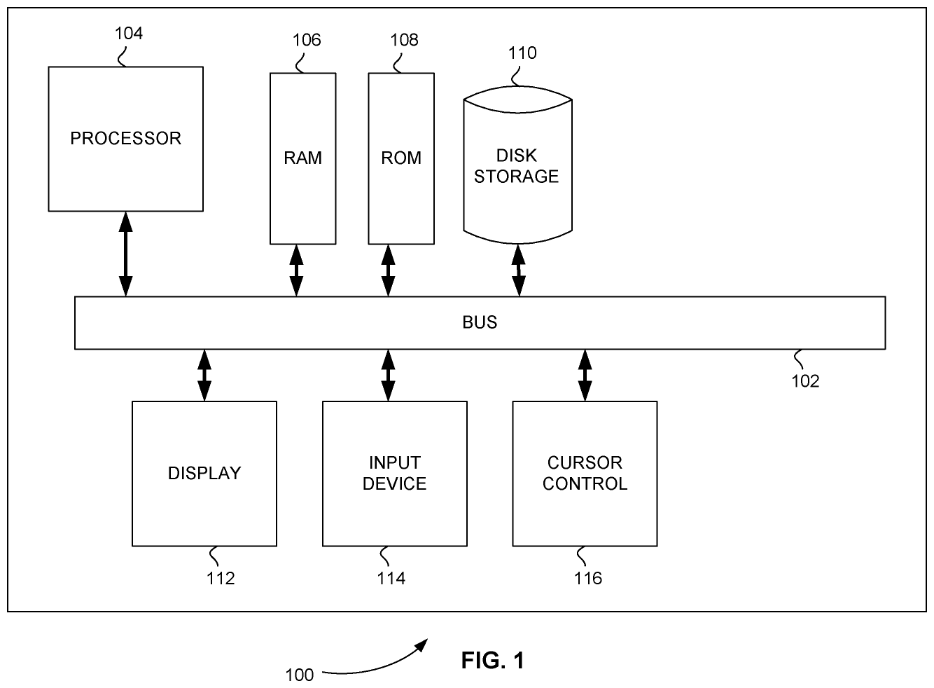

FIG. 1 is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random-access memory (RAM) or other dynamic storage device, coupled to bus 102 for storing instructions to be executed by processor 104. Memory 106 also may be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein. Alternatively, hard-wired circuitry may be used in place of or in combination with software instructions to implement the present teachings. Thus, implementations of the present teachings are not limited to any specific combination of hardware circuitry and software.

The term “computer-readable medium” or “computer program product” as used herein refers to any media that participates in providing instructions to processor 104 for execution. The terms “computer-readable medium” and “computer program product” are used interchangeably throughout this written description. Such a medium may take many forms, including but not limited to, non-volatile media, volatile media, and precursor ion mass selection media. Non-volatile media includes, for example, optical or magnetic disks, such as storage device 110. Volatile media includes dynamic memory, such as memory 106.

Common forms of computer-readable media include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

### Varying Deconvolution Confidence Threshold Per Peak

As described above, a one-way compound identification workflow works well for data from an IDA mass spectrometry method. However, this type of compound identification presents several challenges and disadvantages for data from a DIA mass spectrometry method, often leading to false-positive or false-negative results.

One challenge presented by DIA methods is that the precursor ion (Q1) mass filter is not as specific as the precursor ion mass filter in an IDA method. This reduced specificity results in measured spectra that are a mixture of product ions from multiple different precursor ions. As a result, product ion spectra from DIA methods require that a deconvolution algorithm be performed before a library or database search is done. An exemplary unsupervised clustering algorithm, deconvolution algorithm, linear mapping algorithm, or nonlinear mapping algorithm is PCVG.

Note that PCA is a linear mapping algorithm. In various embodiments a nonlinear mapping algorithm, such as uniform manifold approximation and projection (UMAP) or t-distributed stochastic neighbor embedding (TSNE) clustering can be used. Algorithms like PCA provide powerful mapping to lower dimensions but do not produce clusters. Additional algorithms are need to do clustering (like PCVG does along some angle in PCA space).

Algorithms like PCA typically create 2D or 3D maps of initial variables that are bundled in a cloud if similar. As a result, algorithms like K-mean clustering are needed to find group borders. In various embodiments, a seed from library search as described herein is grown for grouping in PCA space.

Any clustering algorithm has the problem of determining “where is the boundary of the cluster.” Fuzzy clustering is one way to mitigate strict borders and instead of “include”/“exclude,” it has “maybe include.”

For example, the “sigmoidal” family of clustering (often used in Artificial Intelligence algorithms) has continuous output similar to a PCVG angle. A sigmoidal value is produced in one layer before the last layer in the AI architecture and a threshold or voting method is used to produce final class membership result. In various embodiments, the idea of iterative parameter adjustment/library scoring, described below, is used to adjust that threshold or voting decision in order to find the most likely library match.

In summary, PCA is an example of mapping into space where similar variables get arranged in a structure indicative of variable similarity. In PCA, this structure is a line. In various embodiments, however, this structure can be a cloud for an algorithm such as UMAP.

PCVG helps remove product ions from interfering compounds and leads to more accurate purity scores. However, ultimately PCVG calculates a confidence value (based on correlation in principal component (PC) space) that a product ion belongs in the same group.

Often a confidence threshold is applied to obtain a spectrum from PCVG analyzed DIA spectra. This threshold is typically the same for all data. The use of the same confidence threshold for PCVG works well on product ions that are unique for the precursors within an isolation m/z filter window and an LC-peak region. However, shared product ion LC profiles are a linear combination of the corresponding precursor profiles and, therefore, may not pass the confidence threshold. As described above, noise is another factor. Thus, shared product ions between two compounds or very closely eluting compounds can lead to incorrect spectra and poor fit and purity scores.

As a result, additional systems and methods are needed to optimize a parameter of an unsupervised clustering algorithm, deconvolution algorithm, linear mapping algorithm, or nonlinear mapping algorithm, such as the confidence threshold of PCVG, to improve compound identification in mass spectrometry methods like DIA.

### DIA Searching Problem

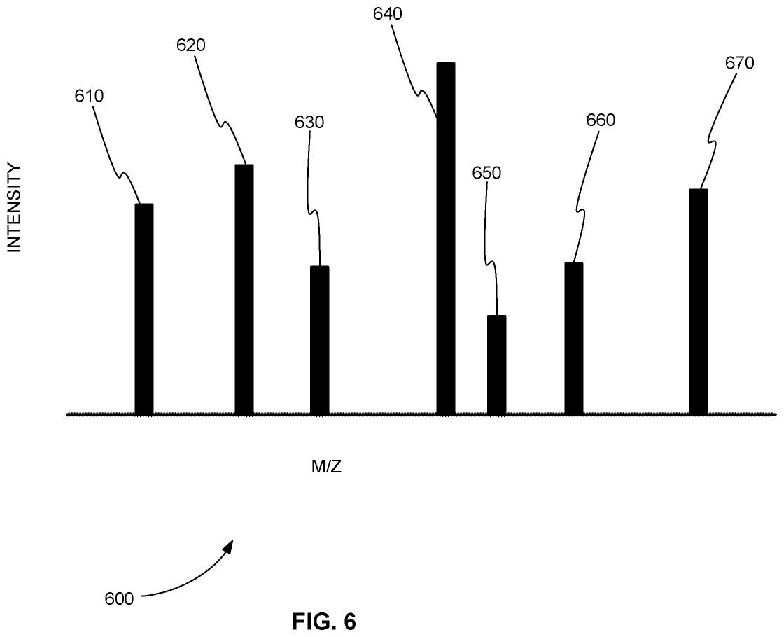

FIG. 6 is an exemplary simplified product ion mass spectrum 600 that is measured during a DIA method, in accordance with various embodiments. Mass spectrum 600 includes seven product ion peaks 610-670. As described above, one of the problems encountered with DIA methods is that the mass spectra produced can include product ions from multiple different precursor ions. Thus, product ion peaks 610-670 may not be from the same precursor ion. As a result, DIA methods are typically performed with an additional technique for separating product ions, such as LC.

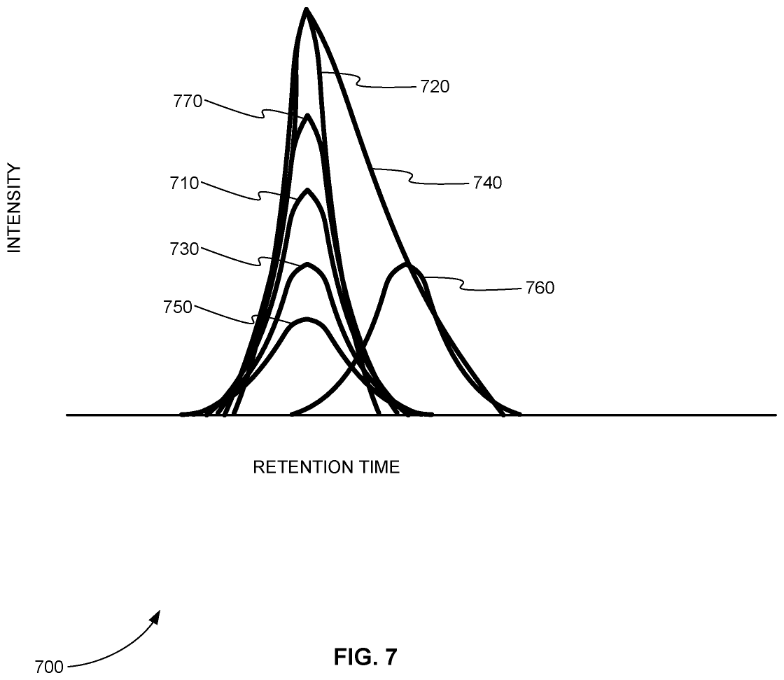

FIG. 7 is an exemplary plot 700 of the XICs of product ions corresponding to the seven peaks shown in FIG. 6, in accordance with various embodiments. XICs 710, 720, 730, 740, 750, 760, and 770 correspond to the same product ions as peaks 610, 620, 630, 640, 650, 660, and 670 of FIG. 6. The five XICs 710, 720, 730, 750, and 770 all clearly have the same retention time, suggesting that corresponding peaks 610, 620, 630, 650, and 670 of FIG. 6 are all from the same compound (precursor ion). XIC 760 appears to have a different retention time suggesting that corresponding peak 660 of FIG. 6 is from a different compound. The retention time of XIC 740 may be slightly different. However, this is due to the fact that it includes an interference with another product ion. In fact, corresponding peak 640 of FIG. 6 is actually from the same compound as the majority of the peaks of FIG. 6 but, from the retention time of XIC 740 of FIG. 7, it appears that XIC 740 is from a different compound.

FIG. 7 shows the typical problem posed by a DIA when attempting to identify compounds. Some product ions may be convolved with other product ions. As a result, product ion spectra from DIA methods require that a deconvolution algorithm be performed before a library or database search is done.

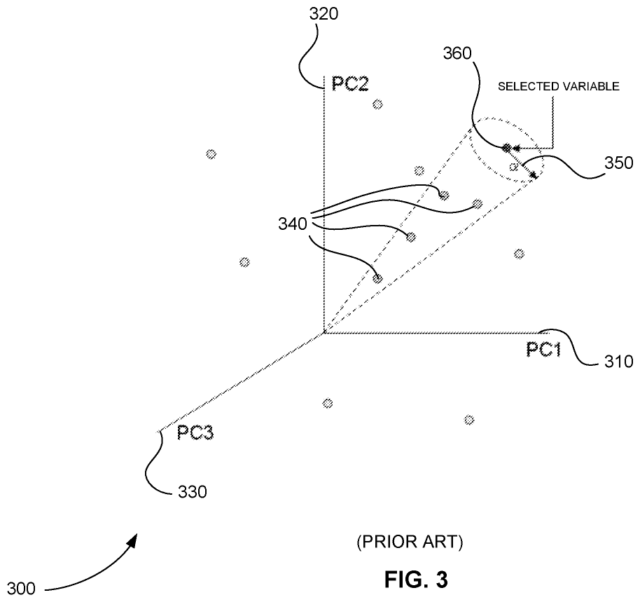

As described above, traditionally deconvolution algorithms like PCVG have been performed with a confidence threshold that is the same for all data. Referring to FIG. 3, this confidence threshold is, for example, inversely related to spatial angle 350. As spatial angle 350 is increased, the confidence threshold is decreased, and, as spatial angle 350 is decreased the confidence threshold is increased. In order to reduce the false-positive rate, the confidence threshold is typically increased. In other words, spatial angle 350 is made to be small.

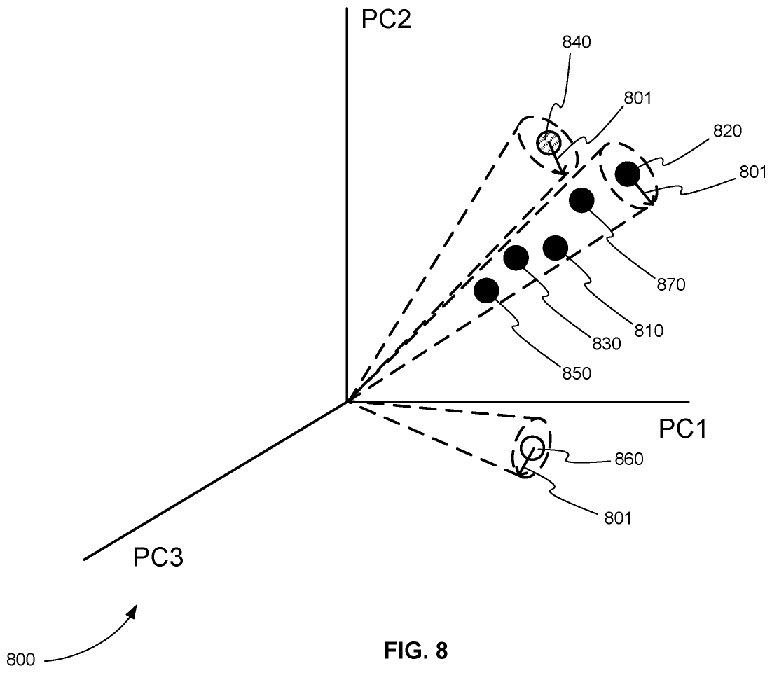

FIG. 8 is an exemplary plot 800 of the product ions of the XICs of FIG. 7 plotted in three-dimensional principal component space after PCVG is performed, in accordance with various embodiments. The XICs of FIG. 7 provided the input to the PCVG algorithm. The confidence threshold or spatial angle 801 was the same for all data.

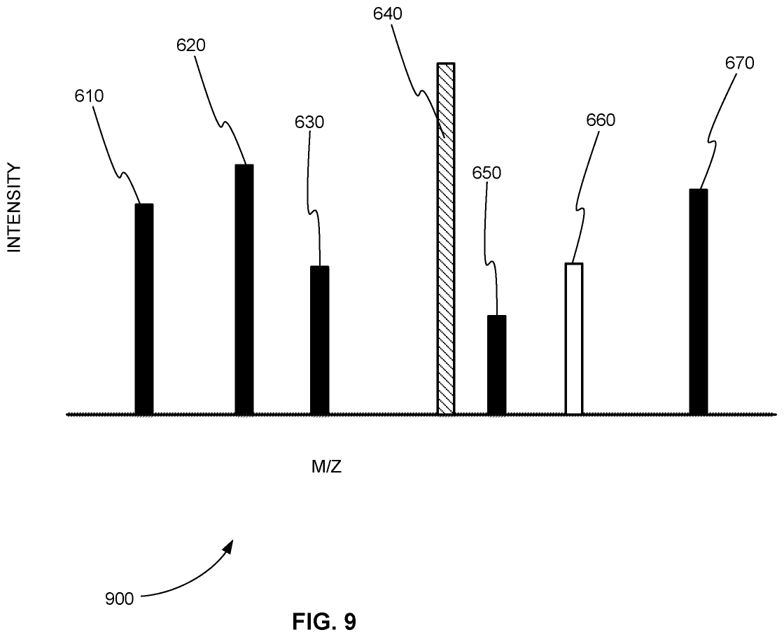

Plot 800 shows that three different groups are found when the same spatial angle 801 is used. The first group includes product ions 810, 820, 830, 850, and 870 corresponding to peaks 610, 620, 630, 650, and 670 of FIG. 6, respectively. The second group includes product ion 840 corresponding to peak 640 of FIG. 6. The third group includes product ion 860 corresponding to peak 660 of FIG. 6. FIG. 9 is an exemplary alternative representation 900 of the simplified product ion mass spectrum of FIG. 6 showing that, after PCVG is performed with the same confidence threshold or spatial angle for all data, two peaks of the mass spectrum are identified originating from different compounds, in accordance with various embodiments. Specifically, in the product ion mass spectrum of FIG. 9, peaks 640 and 660 are shaded differently to show that they originate from different compounds. Traditionally, after PCVG, library or database matching is performed to determine the compound of the measured spectrum.

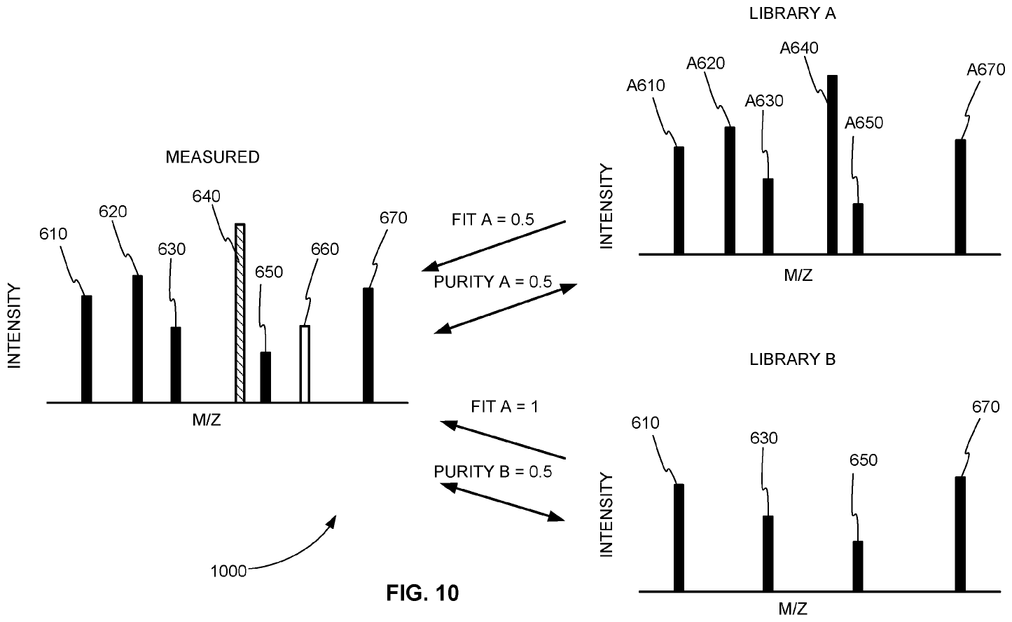

FIG. 10 is a diagram 1000 showing a traditional comparison of the peaks of FIG. 9 after PCVG to two library spectra and showing the fit and purity scores of the comparison, in accordance with various embodiments. Because PCVG determined that peaks 640 and 660 of the measured spectrum were from different compounds, peaks 640 and 660 of the measured spectrum do not participate in the library matching.

Comparing library spectrum A to the measured spectrum shows that only peak A640 of library spectrum A does not match a peak of the measured spectrum. Again, peak 640 of the measured spectrum is treated as belonging to a different compound due to PCVG. The fit score is then 0.5 because of no match for peak A640 of library spectrum A to a peak in the measured spectrum.

Comparing all peaks of both library spectrum A and the measured spectrum also shows again that peak A640 of library spectrum A is not matched. As a result, the purity score is also 0.5.

In contrast, comparing library spectrum B to the measured spectrum shows that all peaks of library spectrum B match a peak of the measured spectrum. The fit score is then a perfect 1.0.

Comparing all peaks of both library spectrum B and the measured spectrum, however, shows a problem. No peaks of library spectrum B match peak 620 of the measured spectrum. As a result, the purity score is reduced to 0.5.

Both library spectra in FIG. 10 have the same purity score. However, library spectrum B has a better fit score than library spectrum A. As a result, in this example, the compound of library spectrum B is identified as the compound of the measured spectrum.

Unfortunately, this identification of the compound of library spectrum B is a false-positive, and the elimination of library spectrum A is a false-negative. The compound of library spectrum A is the correct compound of the measured spectrum. As described above, due to interference with another product ion, peak 640 of the measured spectrum was placed in a different group by PCVG. However, peak 640 is actually a peak of the same compound as the majority of peaks in the measured spectrum.

If peak 640 is included in the matching of the measured spectrum with library spectrum A, both the fit and purity scores are 1. If peak 640 is included in the matching of the measured spectrum with library B, the fit score is still 1, but the purity score is further reduced to 0.4 due to the inclusion of peak 640 in the measured spectrum. Thus, the compound of library spectrum A is the correct compound of the measured spectrum.

Consequently, FIGS. 8-10 show how the use of the same confidence threshold for all data can result in false-positive and false-negative results.

### Solution—Adjusting PCVG Parameters

In various embodiments, both false-positive and false-negative results are reduced by adjusting PCVG (or deconvolution/clustering) grouping parameters iteratively based on library scores. For example, a library score is used to reassign peaks from a “convolved peaks group” to contributing pure LC peaks groups. Typically, if the contribution of an interfering peak to both precursors is comparable (>10%), it is most likely that the interfering peak is put in its own group.

In various embodiments, both false-positive and false-negative results are reduced specifically by adjusting the confidence threshold of the grouping of the deconvolution algorithm based on the fit score contribution. Specifically, first, using only the fit score, the measured spectrum is compared to each spectrum of the library. All library spectra with a fit score above a certain fit score threshold are included in a set of library spectra that are potentially correct matching spectra.

For each library spectrum of the set of library spectra, PCVG grouping is recalculated where each product ion in PC space is grouped based on a confidence threshold that depends on how much the product ion contributed to the fit score of the library spectrum. Product ions that are important to the fit score have a lower confidence threshold than normal. A lower confidence threshold translates to an increased spatial angle. A group of peaks of the measured spectrum found from this type of PCVG is then compared to peaks of the library spectrum using a purity score.

The compound corresponding to the library spectrum of the set of library spectra that has the highest purity score is identified as the compound of the measured spectrum.

Note that the confidence threshold is not adjusted for particular fragment but for the corresponding PCVG group assembly. The confidence threshold is applied to all fragments. This lets the fragment of interest get included but it might include other fragments that happen to fall within that new, larger angle. If the spectrum defined by the PCVG group, formed of all fragments from that newly determine angle, has higher score then previous spectra defined by narrower angle, the process is continued for the next “highly contributing” fragment to the fit score. This continues as long as the score is getting higher. Once the angle is increased to include too many fragments with not that similar LC profiles (large angle), the process is stopped. The best scoring pair (spectra defined by PCVG group, PCVG grouping angle threshold) is recorded as the best match to that library spectrum. This is repeated for all candidate library spectra.

In various embodiments, this group recalculation starts from a default PCVG grouping result. In various alternative embodiments, traditional PCVG is not done. Instead, PCA is performed. For given library spectrum, the highest contributing fragment is used as a seed for grouping in PC space, with angle θ. The PCVG spectrum consists of that fragment only. The score is then calculated using just that fragment to start.

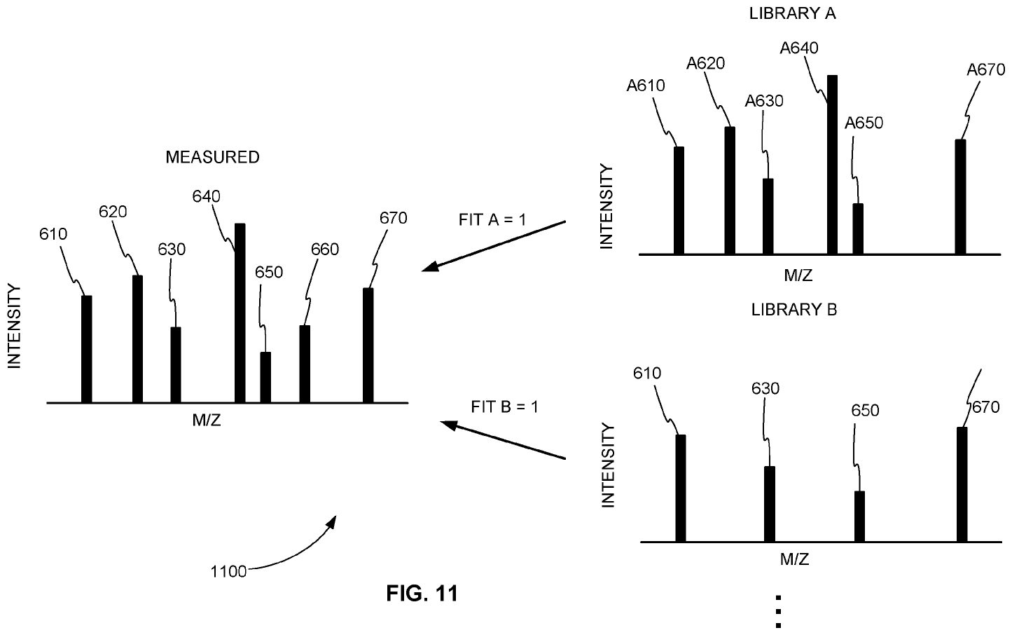

FIG. 11 is a diagram 1100 showing a comparison of the raw peaks of the measured spectrum of FIG. 6 before PCVG to the peaks of the spectra of a library using only the fit score, in accordance with various embodiments. As described above, in various embodiments, using only the fit score, the raw measured spectrum is compared to each spectrum of the library.

FIG. 11 shows the raw measured spectrum of FIG. 6 being compared to the spectra of a library using only the fit score. Both library spectrum A and library spectrum B have perfect fit scores when compared to the raw measured spectrum of FIG. 6. As a result, both library spectrum A and library spectrum B have a fit score above a predetermined fit score threshold and are included in the set of library spectra that are potentially correct matching spectra.

For each spectrum of the set of library spectra that are potentially correct matching spectra, PCVG grouping is recalculated. For example, PCVG grouping is recalculated separately for library spectrum A and library spectrum B. For each spectrum of the set of library spectra, the confidence threshold used for an individual product ion clustering is varied based on the contribution of that individual product ion to the fit score.

For example, for library spectrum A of FIG. 11, peak 640 of the raw measured spectrum provides a significant contribution to the fit score of 1. In other words, peak A640 of library spectrum matching peak 640 of the raw measured spectrum contributes significantly to the fit score of 1. As a result, peak 640 of the raw measured spectrum may be given a contribution score of 73%, for example. A contribution score above a predetermined contribution score threshold (e.g., 60%) then reduces the confidence threshold for PCVG grouping. For example, due to its high contribution score, the confidence threshold for PCVG grouping is reduce to include the product ion of peak 640 of the raw measured spectrum. In other words, the spatial angle for the PCVG group corresponding to library spectrum A is increased to include product ion of peak 640 of the raw measured spectrum. As a result, PCVG then finds peak 640 of the raw measured spectrum to be in the same group as the majority of peaks in the measured spectrum.

By including peak 640 in the group, additional ions might be included. Any peaks falling within that increased angle will be included and will affect the library score. In this way, a continual increase in the angle to include less significant ions in spectrum A causes the library score and purity score to decrease. Essentially, peaks often cannot be added without penalty. Also, the penalty is different for each different library spectrum iterative adjustment of PCVG grouping. Calculating the penalty with each iterative adjustment of PCVG grouping produces the peaks that most likely match.

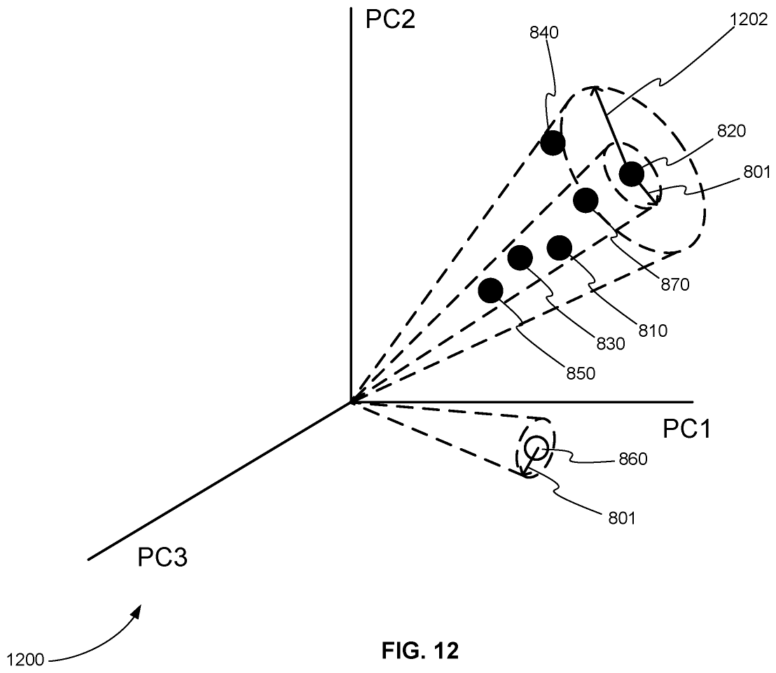

FIG. 12 is an exemplary plot 1200 of the product ions of the XICs of FIG. 7 plotted in three-dimensional principal component space showing how the confidence threshold is lowered during PCVG grouping to include a highly contributing product ion to the fit score of a particular library spectrum, in accordance with various embodiments. In plot 1200, for product ion 840, the confidence threshold is lowered. In other words, a larger spatial angle 1202 is used rather than the normal spatial angle 801. The confidence threshold for the PCVG group corresponding to library spectrum A is lowered due to the contribution of product ion 840's peak 640 in FIG. 11 to the fit score of library spectrum A. Thus, the confidence threshold for PCVG grouping of FIG. 12 is lowered only for library spectrum A of FIG. 11.

Decreasing the confidence threshold for product ion 840 of FIG. 12 and increasing the spatial angle to spatial angle 1202, places product ion in the same group as product ions 810, 820, 830, 850, and 870. In other words, PCVG grouping now finds product ion 840 to be from the same compound (precursor ion) as product ions 810, 820, 830, 850, and 870.

Note, again, that some additional ion might also fall within that angle (not plotted in the current figure) but that ion contribution to the purity score (negative contribution) is much smaller than positive contribution of product ion 840. This is why the most contributing missing ion is chosen to start with.

Returning to FIG. 11, in contrast, peak 640 of the raw measured spectrum does not contribute to the fit score of library spectrum B. As a result, peak 640 of the raw measured spectrum may be given a contribution score of 0%, for example, for library spectrum B. Due to this low contribution score, the confidence threshold for the product ion of peak 640 of the raw measured spectrum in the PCVG performed for creating group corresponding to library spectrum B is not reduced. In other words, the spatial angle for the product ion of peak 640 of the raw measured spectrum in PCVG is the normal or default spatial angle. As a result, PCVG for library spectrum B finds peak 640 of the raw measured spectrum to be in a separate group just as in FIG. 8.

Note that, in various embodiments, the entire PCVG analysis is not performed for each library spectrum. Instead a default PCVG is performed. Then, for each library spectrum selected as described above, the PCVG group that has largest number of high contributing peaks for the corresponding library spectrum is found. Then, angle adjustment is performed iteratively to try to get more high contributing fragments, until the purity score starts to degrade.

After the default PCVG analysis is performed, for each library spectrum of the set of library spectra, the first group of peaks is compared to the corresponding library spectrum. For example, the peaks of the measured spectrum of FIGS. 6, 610, 620, 630, 640, 650, and 670 corresponding to product ions 810, 820, 830, 840, 850, and 870, respectively, of the first group of FIG. 12 are compared to library spectrum A of FIG. 11. Similarly, the peaks of the measured spectrum of FIGS. 6, 610, 620, 630, 650, and 670 corresponding to product ions 810, 820, 830, 850, and 870, respectively, of the first group of FIG. 8 are compared to library spectrum B of FIG. 11.

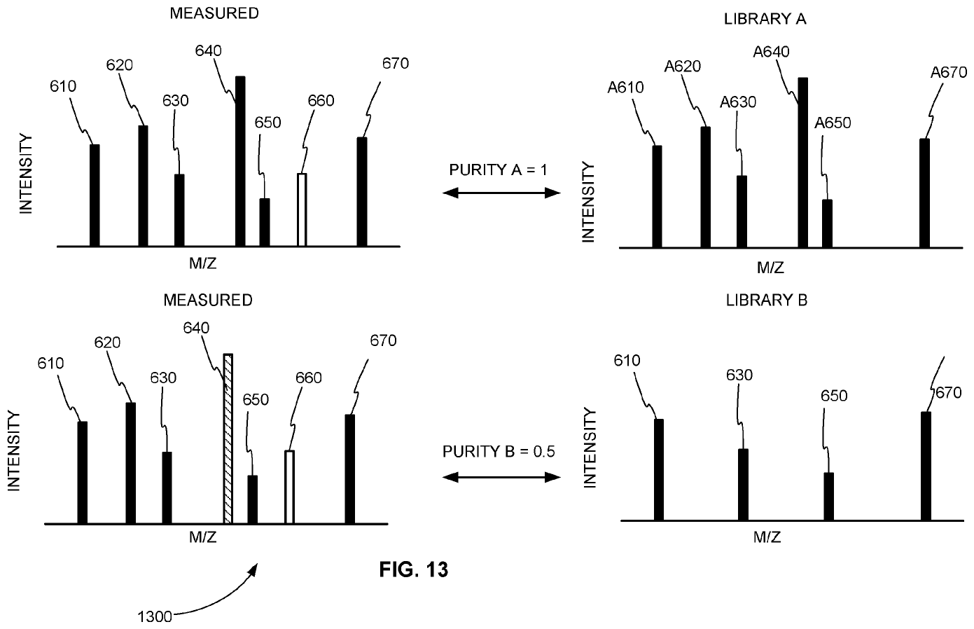

FIG. 13 is a diagram 1300 showing how correlated peaks found through PCVG grouping for a particular library spectrum are then compared to that library spectrum using only a purity score, in accordance with various embodiments. FIG. 13 shows that, for library spectrum A, recalculated PCVG grouping found that only peak 660 of the measured spectrum should be excluded. As a result, the comparison of the measured spectrum excluding peak 660 to library spectrum A produced a purity score of 1.

In contrast, FIG. 13 shows that, for library spectrum B, recalculated PCVG grouping found that both peaks 640 and 660 of the measured spectrum should be excluded. As a result, the comparison of the measured spectrum to library spectrum B excluding peaks 640 and 660 produced a purity score of 0.5.

In comparison with FIG. 10, FIG. 13 now shows that library spectrum A has a higher purity score. The compound corresponding to the library spectrum of the set of library spectra that has the highest purity score is identified as the compound of the measured spectrum. Thus, the compound corresponding to library A is identified as the compound of the measured spectrum.

As described above, the compound corresponding to library A is the correct answer. Thus, FIGS. 11-13 show that modifying the PCVG grouping per library spectrum based on the fit score has provided the correct result.

### System for Identifying a Library Mass Spectrum

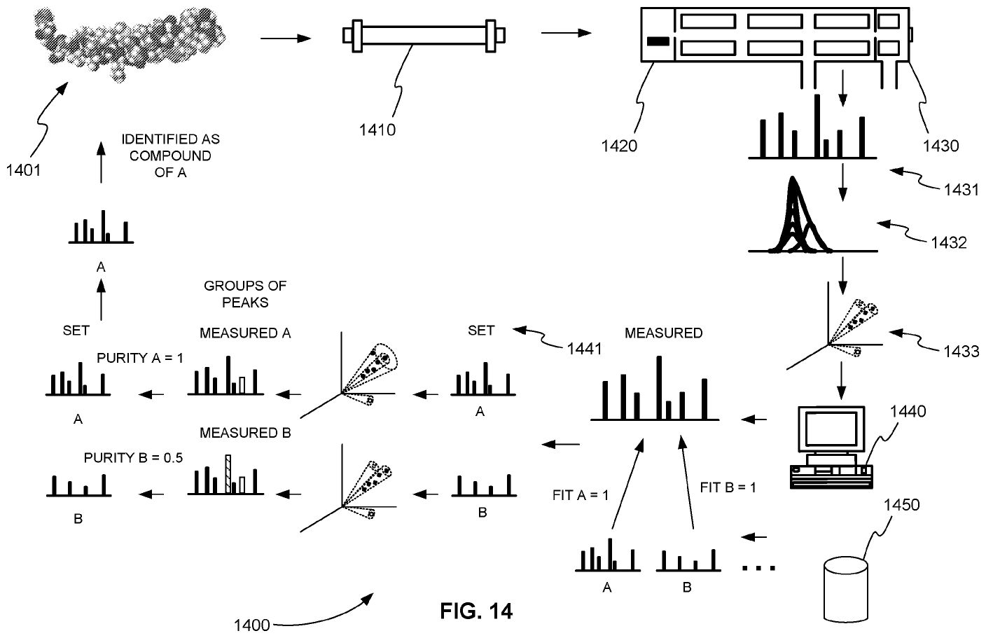

FIG. 14 is a schematic diagram 1400 of a system for identifying a library mass spectrum of a known compound that matches a measured mass spectrum, in accordance with various embodiments. The system includes processor 1440. Processor 1440 can be, but is not limited to, a controller, a computer, a microprocessor, the computer system of FIG. 1, or any device capable of analyzing data. Processor 1440 can also be any device capable of sending and receiving control signals and data.

In general, processor 1440 receives measured mass spectrum 1431 and additional intensity data 1432 of a known or unknown compound 1401. Processor 1440 identifies a set 1441 of mass spectra of known compounds matching measured spectrum 1431 using a first score and calculates a group 1433 of related peaks of the measured spectrum. For each spectrum of the set, processor 1440 recalculates group 1433 using the additional data and compares peaks of group 1433 to measured spectrum 1431 using a second score. Finally, processor 1440 identifies at least one spectrum of the set with the highest second score as a match for compound 1401. More specifically, processor 1440 receives measured mass spectrum 1431 and additional intensity data 1432 for ions of the measured mass spectrum as a function of mass-to-charge ratio (m/z) and at least one additional dimension from a mass spectrometry method. Processor 1440 compares peaks of measured mass spectrum 1431 to peaks of each of a plurality of library mass spectra of different known compounds. Processor 1440 calculates a first score for how well peaks of each library mass spectrum match peaks of measured mass spectrum 1431. Processor 1440 identifies set 1441 of library mass spectra 1441 where each mass spectrum of set 1441 has a score above a first score threshold. In various embodiments, the first score is a fit score. Processor 1440 calculates a group 1433 of related peaks of the measured mass spectrum using an unsupervised clustering algorithm, deconvolution algorithm, linear mapping algorithm, or nonlinear mapping algorithm.

For each library mass spectrum of the set, processor 1440 recalculates the group of related peaks of the measured mass spectrum. The recalculation uses the additional intensity data to lower a threshold for selection in the group for a peak of the measured mass spectrum if a matching peak of the library mass spectrum contributed to the first score of the library mass spectrum above a contribution threshold amount. A corresponding group of related peaks of the measured mass spectrum is produced for each library mass spectrum of the set 1441.

In various embodiments, this lowering of the threshold for selection in the group is controlled. For example, for each peak added by lowering the threshold, library scores (e.g., both fit and purity scores) are calculated. If a score improves, the lowering of the threshold continues. However, at some point, too many unwanted peaks will be added and that score will go down. As a result, there is a maximum score that can be achieved for each library spectrum. The score that is largest among them all, indicates the spectrum with the best match.

For each library mass spectrum of the set, processor 1440 compares peaks of the corresponding group of the library mass spectrum to peaks of the library mass spectrum and calculates a second score for how well all peaks of the corresponding group of the library mass spectrum match all peaks of the library mass spectrum. Processor 1440 identifies at least one library mass spectrum of the set with the highest second score. The compound of the at least one library mass spectrum is then identified as the compound of the measured spectrum. In various embodiments, the second score is a purity score.

In various embodiments, the system of FIG. 14 further includes mass spectrometer 1430 that measures mass spectrum 1431 and additional intensity data 1432 and sends mass spectrum 1431 and additional intensity data 1432 to processor 1440. Ion source device 1420 of mass spectrometer 1430 ionizes separated fragments of compound 1401 or only compound 1401, producing an ion beam. Ion source device 1420 is controlled by processor 1440, for example. Ion source device 1420 is shown as a component of mass spectrometer 1430. In various alternative embodiments, ion source device 1420 is a separate device. Ion source device 1420 can be, but is not limited to, an electrospray ion source (ESI) device or a chemical ionization (CI) source device such as an atmospheric pressure chemical ionization source (APCI) device or an atmospheric pressure photoionization (APPI) source device.

Mass spectrometer 1430 mass analyzes product ions of compound 1401 or selects and fragments compound 1401 and mass analyzes product ions of compound 1401 from the ion beam at a plurality of different times. Mass spectrum 1431 and additional data 1432 are produced for compound 1401. Mass spectrometer 1430 is controlled by processor 1440, for example.

In the system of FIG. 14, mass spectrometer 1430 is shown as a triple quadrupole device. One of ordinary skill in the art can appreciate that any component of mass spectrometer 1430 can include other types of mass spectrometry devices including, but not limited to, ion traps, orbitraps, time-of-flight (TOF) devices, ion mobility devices, or Fourier transform ion cyclotron resonance (FT-ICR) devices.

In various embodiments, the system of FIG. 14 further includes additional device 1410 affects compound 1401 providing the at least one additional dimension. As shown in FIG. 14, additional device 1410 is an LC device and the at least one additional dimension provided is retention time. In various alternative embodiments, additional device 1410 can be, but is not limited to, a gas chromatography (GC) device, capillary electrophoresis (CE) device, an ion mobility spectrometry (IMS) device, or a differential mobility spectrometry (DMS) device. In still further embodiments, additional device 1410 is not used and the at least one additional dimension provided is retention time is precursor ion m/z and is provided by mass spectrometer 1430 operating in a precursor ion scanning mode.

In various embodiments, the system of FIG. 14 further includes database or library 1450 that provides the library of mass spectra to processor 1440.

### Method for Identifying a Library Mass Spectrum

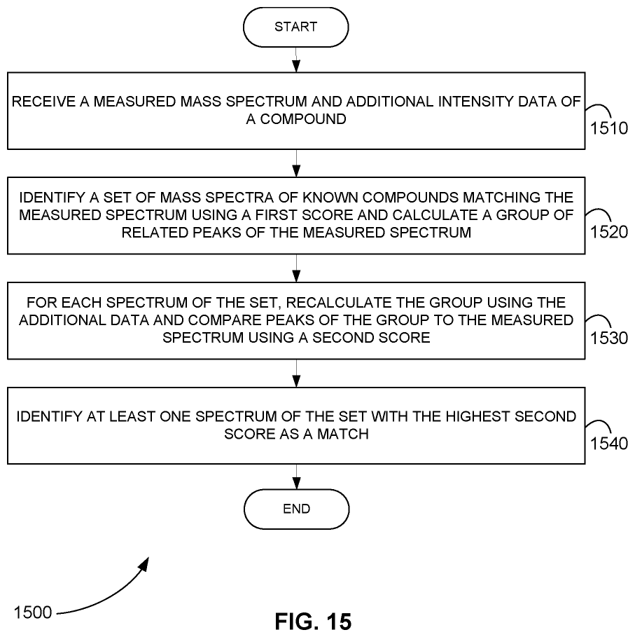

FIG. 15 is an exemplary flowchart showing a method 1500 for identifying a library mass spectrum of a known compound that matches a measured mass spectrum, in accordance with various embodiments.

In step 1510 of method 1500, a measured mass spectrum and additional intensity data of a compound are received.

In step 1520, a set of mass spectra of known compounds matching the measured spectrum is identified using a first score and a group of related peaks of the measured spectrum is calculated.

In step 1530, for each spectrum of the set, the group is recalculated using the additional data and peaks of the group are compared to the measured spectrum using a second score.

In step 1540, identify at least one spectrum of the set with the highest second score as a match.

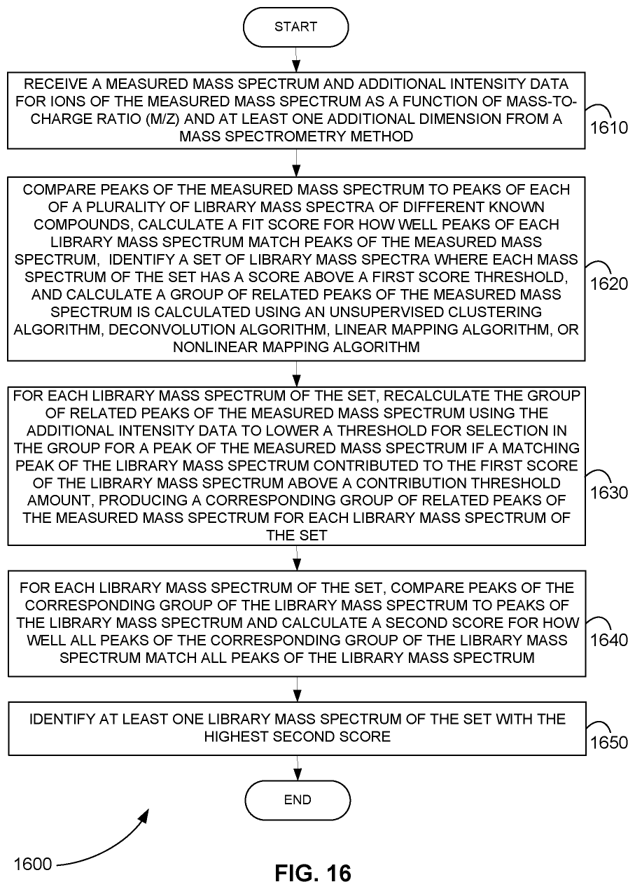

FIG. 16 is an exemplary flowchart showing a detailed method 1600 for identifying a library mass spectrum of a known compound that matches a measured mass spectrum, in accordance with various embodiments.

In step 1610 of method 1600, a measured mass spectrum and additional intensity data for ions of the measured mass spectrum as a function of mass-to-charge ratio (m/z) and at least one additional dimension are received from a mass spectrometry method.

In step 1620, peaks of the measured mass spectrum are compared to peaks of each of a plurality of library mass spectra of different known compounds, a first score is calculated for how well peaks of each library mass spectrum match peaks of the measured mass spectrum, a set of library mass spectra is identified where each mass spectrum of the set has a score above a first score threshold, and a group of related peaks of the measured mass spectrum is calculated using an unsupervised clustering algorithm, deconvolution algorithm, linear mapping algorithm, or nonlinear mapping algorithm. In various embodiments, the first score is a fit score.

In step 1630, for each library mass spectrum of the set, the group of related peaks of the measured mass spectrum is recalculated. using an unsupervised clustering algorithm, deconvolution algorithm, linear mapping algorithm, or nonlinear mapping algorithm. The recalculation uses the additional intensity data to lower a threshold for selection in the group for a peak of the measured mass spectrum if a matching peak of the library mass spectrum contributed to the first score of the library mass spectrum above a contribution threshold amount. A corresponding group of related peaks of the measured mass spectrum is produced for each library mass spectrum of the set.

In step 1640, for each library mass spectrum of the set, peaks of the corresponding group of the library mass spectrum are compared to peaks of the library mass spectrum and a second score is calculated for how well all peaks of the corresponding group of the library mass spectrum match all peaks of the library mass spectrum.

In step 1650, at least one library mass spectrum of the set with the highest second score is identified.

In various embodiments, the first score is a fit score and the second score is a purity score.

In various embodiments, the unsupervised clustering algorithm, deconvolution algorithm, linear mapping algorithm, or nonlinear mapping algorithm includes PCVG.

In various embodiments, wherein the at least one additional dimension includes retention time.

In various embodiments, the at least one additional dimension includes a precursor ion m/z of a method in which the precursor ion m/z range is scanned.

In various embodiments, the at least one additional dimension includes a compensation voltage (CoV) of a differential mobility spectrometry (DMS) device.

In various embodiments, the at least one additional dimension includes a drift time or a collision cross-section of an ion mobility spectrometry (IMS) device.

In various embodiments, the mass spectrometry method includes a DIA method.

In various embodiments, as described above, the lowering of the threshold for selection in the group is controlled. Specifically, for each peak added to a group of related peaks of the measured mass spectrum for each library mass spectrum of the set, one or more of the first score and the second score are recalculated and compared to one or more of a previous first score and a previous second score to determine if a threshold lowering limit is reached. Again, if a score improves, the lowering of the threshold continues. However, at some point, too many unwanted peaks will be added and that score will go down. As a result, there is a maximum score that can be achieved for each library spectrum. The score that is largest among them all, indicates the spectrum with the best match.

### Computer Program Product for Identifying a Library Mass Spectrum

In various embodiments, a computer program product includes a non-transitory tangible computer-readable storage medium whose contents include a program with instructions being executed on a processor so as to perform a method for identifying a library mass spectrum of a known compound that matches a measured mass spectrum. This method is performed by a system that includes one or more distinct software modules.

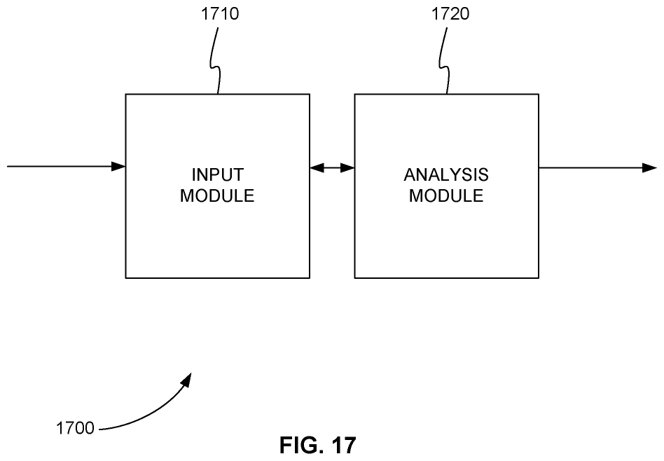

FIG. 17 is a schematic diagram of a system 1700 that includes one or more distinct software modules and that performs a method for identifying a library mass spectrum of a known compound that matches a measured mass spectrum, in accordance with various embodiments. System 1700 includes input module 1710 and analysis module 1720.

In general, input module 1710 receives a measured mass spectrum and additional intensity data of a compound. Analysis module 1720 identifies a set of mass spectra of known compounds matching the measured spectrum using a first score and calculates a group of related peaks of the measured spectrum. For each spectrum of the set, analysis module 1720 recalculates the group using the additional data and compares peaks of the group to the measured spectrum using a second score. Analysis module 1720 identifies at least one spectrum of the set with the highest second score as a match.

More specifically, input module 1710 receives a measured mass spectrum and additional intensity data for ions of the measured mass spectrum as a function of mass-to-charge ratio (m/z) and at least one additional dimension from a mass spectrometry method.

Analysis module 1720 compares peaks of the measured mass spectrum to peaks of each of a plurality of library mass spectra of different known compounds. Analysis module 1720 calculates a first score for how well peaks of each library mass spectrum match peaks of the measured mass spectrum. Analysis module 1720 identifies a set of library mass spectra where each mass spectrum of the set has a score above a first score threshold. Analysis module 1720 calculates a group of related peaks of the measured mass spectrum using an unsupervised clustering algorithm, deconvolution algorithm, linear mapping algorithm, or nonlinear mapping algorithm.

For each library mass spectrum of the set, analysis module 1720 recalculates the group of related peaks of the measured mass spectrum. The recalculation uses the additional intensity data to lower a threshold for selection in the group for a peak of the measured mass spectrum if a matching peak of the library mass spectrum contributed to the first score of the library mass spectrum above a contribution threshold amount. A corresponding group of related peaks of the measured mass spectrum is produced for each library mass spectrum of the set.

For each library mass spectrum of the set, analysis module 1720 compares peaks of the corresponding group of the library mass spectrum to peaks of the library mass spectrum and calculates a second score for how well all peaks of the corresponding group of the library mass spectrum match all peaks of the library mass spectrum.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

1. A method for identifying a library mass spectrum of a known compound that matches a measured mass spectrum, comprising:
receiving a measured mass spectrum and additional intensity data of a compound;
identifying a set of mass spectra of known compounds matching the measured spectrum using a first score and calculating a group of related peaks of the measured spectrum;
for each spectrum of the set, recalculating the group using the additional data and comparing peaks of the group to the measured spectrum using a second score; and
identifying at least one spectrum of the set with the highest second score as a match.

2. The method of claim 1,
wherein receiving a measured mass spectrum and additional intensity data includes receiving a measured mass spectrum and additional intensity data for ions of the measured mass spectrum as a function of mass-to-charge ratio (m/z) and at least one additional dimension from a mass spectrometry method;
wherein identifying a set of mass spectra and calculating a group of related peaks includes comparing peaks of the measured mass spectrum to peaks of each of a plurality of library mass spectra of different known compounds, calculating a first score for how well peaks of each library mass spectrum match peaks of the measured mass spectrum, identifying a set of library mass spectra where each mass spectrum of the set has a score above a first score threshold, and calculating a group of related peaks of the measured mass spectrum using an unsupervised clustering algorithm, deconvolution algorithm, linear mapping algorithm, or nonlinear mapping algorithm;
wherein, for each spectrum of the set, recalculating the group includes, for each library mass spectrum of the set, recalculating the group of related peaks of the measured mass spectrum using the additional intensity data to lower a threshold for selection in the group for a peak of the measured mass spectrum if a matching peak of the each library mass spectrum contributed to the first score of the each library mass spectrum above a contribution threshold amount, producing a corresponding group of related peaks of the measured mass spectrum for each library mass spectrum of the set;
wherein, for each spectrum of the set, comparing peaks of the group includes, for each library mass spectrum of the set, comparing peaks of the corresponding group of the each library mass spectrum to peaks of the each library mass spectrum and calculating a second score for how well all peaks of the corresponding group of the each library mass spectrum match all peaks of the each library mass spectrum; and
wherein identifying at least one spectrum of the set with the highest second score as a match includes identifying at least one library mass spectrum of the set with the highest second score.

3. The method of claim 2, wherein the unsupervised clustering algorithm, deconvolution algorithm, linear mapping algorithm, or nonlinear mapping algorithm comprises principal component analysis with variable grouping (PCVG).

4. The method of claim 2, wherein the at least one additional dimension comprises retention time or the at least one additional dimension comprises a precursor ion m/z.

5. The method of claim 2, wherein the at least one additional dimension comprises a compensation voltage (CoV) of a differential mobility spectrometry (DMS) device.

6. The method of claim 2, wherein the at least one additional dimension comprises a drift time or a collision cross-section of an ion mobility spectrometry (IMS) device.

7. The method of claim 2, wherein the mass spectrometry method comprises a data-independent acquisition (DIA) method.

8. The method of claim 2, wherein the first score comprises a fit score and the second score comprises a purity score.

9. The method of claim 2, wherein for each peak added to a group of related peaks of the measured mass spectrum for each library mass spectrum of the set, one or more of the first score and the second score are recalculated and compared to one or more of a previous first score and a previous second score to determine if a threshold lowering limit is reached.

10. A computer program product, comprising a non-transitory tangible computer-readable storage medium whose contents cause a processor to perform a method for identifying a library mass spectrum of a known compound that matches a measured mass spectrum, the method comprising:
providing a system, wherein the system comprises one or more distinct software modules, and wherein the distinct software modules comprise an input module and an analysis module;
receiving a measured mass spectrum and additional intensity data of a compound using the input module;
identifying a set of mass spectra of known compounds matching the measured spectrum using a first score and calculating a group of related peaks of the measured spectrum using the analysis module;
for each spectrum of the set, recalculating the group using the additional data and comparing peaks of the group to the measured spectrum using a second score using the analysis module; and
identifying at least one spectrum of the set with the highest second score as a match using the analysis module.

11. The computer program product of claim 10,
wherein receiving a measured mass spectrum and additional intensity data includes receiving a measured mass spectrum and additional intensity data for ions of the measured mass spectrum as a function of mass-to-charge ratio (m/z) and at least one additional dimension from a mass spectrometry method;
wherein identifying a set of mass spectra and calculating a group of related peaks includes comparing peaks of the measured mass spectrum to peaks of each of a plurality of library mass spectra of different known compounds, calculating a first score for how well peaks of each library mass spectrum match peaks of the measured mass spectrum, identifying a set of library mass spectra where each mass spectrum of the set has a score above a first score threshold, and calculating a group of related peaks of the measured mass spectrum using an unsupervised clustering algorithm, deconvolution algorithm, linear mapping algorithm, or nonlinear mapping algorithm;
wherein, for each spectrum of the set, recalculating the group includes, for each library mass spectrum of the set, recalculating the group of related peaks of the measured mass spectrum using the additional intensity data to lower a threshold for selection in the group for a peak of the measured mass spectrum if a matching peak of the each library mass spectrum contributed to the first score of the each library mass spectrum above a contribution threshold amount, producing a corresponding group of related peaks of the measured mass spectrum for each library mass spectrum of the set;
wherein, for each spectrum of the set, comparing peaks of the group includes, for each library mass spectrum of the set, comparing peaks of the corresponding group of the each library mass spectrum to peaks of the each library mass spectrum and calculating a second score for how well all peaks of the corresponding group of the each library mass spectrum match all peaks of the each library mass spectrum; and
wherein identifying at least one spectrum of the set with the highest second score as a match includes identifying at least one library mass spectrum of the set with the highest second score.

12. The computer program product of claim 11, wherein the unsupervised clustering algorithm, deconvolution algorithm, linear mapping algorithm, or nonlinear mapping algorithm comprises principal component analysis with variable grouping (PCVG).

13. The computer program product of claim 11, wherein the first score comprises a fit score and the second score comprises a purity score.

14. A system for identifying a library mass spectrum of a known compound that matches a measured mass spectrum, comprising:
a processor that
receives a measured mass spectrum and additional intensity data of a compound;
identifies a set of mass spectra of known compounds matching the measured spectrum using a first score and calculates a group of related peaks of the measured spectrum;
for each spectrum of the set, recalculates the group using the additional data and compares peaks of the group to the measured spectrum using a second score; and
identifies at least one spectrum of the set with the highest second score as a match.

15. The system of claim 14,
wherein the processor receives the measured mass spectrum and additional intensity data by receiving a measured mass spectrum and additional intensity data for ions of the measured mass spectrum as a function of mass-to-charge ratio (m/z) and at least one additional dimension from a mass spectrometry method;
wherein the processor identifies a set of mass spectra and calculating a group of related peaks by comparing peaks of the measured mass spectrum to peaks of each of a plurality of library mass spectra of different known compounds, calculating a first score for how well peaks of each library mass spectrum match peaks of the measured mass spectrum, identifying a set of library mass spectra where each mass spectrum of the set has a score above a first score threshold, and calculating a group of related peaks of the measured mass spectrum using an unsupervised clustering or deconvolution algorithm;
wherein, for each spectrum of the set, the processor recalculates the group by, for each library mass spectrum of the set, recalculating the group of related peaks of the measured mass spectrum using the additional intensity data to lower a threshold for selection in the group for a peak of the measured mass spectrum if a matching peak of the each library mass spectrum contributed to the first score of the each library mass spectrum above a contribution threshold amount, producing a corresponding group of related peaks of the measured mass spectrum for each library mass spectrum of the set;
wherein, for each spectrum of the set, the processor compares peaks of the group by, for each library mass spectrum of the set, comparing peaks of the corresponding group of the each library mass spectrum to peaks of the each library mass spectrum and calculating a second score for how well all peaks of the corresponding group of the each library mass spectrum match all peaks of the each library mass spectrum; and
wherein the processor identifies at least one spectrum of the set with the highest second score as a match by identifying at least one library mass spectrum of the set with the highest second score.
